(19) United States
(12) Patent Application Publication
(10) Pub. No.: US 2025/0258264 A1
(43) Pub. Date: Aug. 14, 2025

(54) Magnetic Resonance System Comprising a Magnetic Resonance Apparatus and an Elastography Apparatus

(71) Applicant: Siemens Healthineers AG, Forchheim (DE)

(72) Inventor: Johann Sukkau, Herzogenaurach (DE)

(73) Assignee: Siemens Healthineers AG, Forchheim (DE)

(21) Appl. No.: 19/051,530

(22) Filed: Feb. 12, 2025

(30) Foreign Application Priority Data
Feb. 13, 2024 (EP) 24157310.4

(51) Int. Cl. G01R 33/563; G01R 33/34; G01R 33/38

(52) U.S. Cl. CPC G01R 33/56358; G01R 33/34061; G01R 33/3808

(57) ABSTRACT

The disclosure is based on a magnetic resonance system comprising a magnetic resonance apparatus with a scanner unit having a main magnet, a gradient coil unit, and a radio-frequency coil unit; a patient receiving region at least partially surrounded by the scanner unit, and an elastography apparatus embodied to excite regions of interest of a patient during a magnetic resonance elastography examination on the patient, comprising a vibration unit, a magnetic resonance-compatible drive unit, and a transmission unit for transmitting a drive torque generated by the magnetic resonance-compatible drive unit to the vibration unit. The magnetic resonance-compatible drive unit has a magnetic resonance-compatible motor.

### CROSS-REFERENCE TO RELATED APPLICATIONS

The present application claims priority to and the benefit of European patent application no. EP 24157310.4, filed on Feb. 13, 2024, the contents of which are incorporated herein by reference in their entirety.

### TECHNICAL FIELD

The present disclosure relates to a magnetic resonance system comprising a magnetic resonance apparatus with a scanner unit having a main magnet, a gradient coil unit, and a radio-frequency antenna unit, and a patient receiving region at least partially surrounded by the scanner unit. Moreover, the magnetic resonance system comprises an elastography apparatus embodied to excite regions of interest of a patient during a magnetic resonance elastography examination on the patient comprising a vibration unit, a magnetic resonance-compatible drive unit, and a transmission unit for transmitting a drive torque generated by the magnetic resonance-compatible drive unit to the vibration unit.

### BACKGROUND

Healthy tissue and tumor tissue or diseased tissue, for example a liver affected by liver fibrosis, exhibit different vibration properties and/or different excitation behavior under vibration excitation. In elastography, diagnostic imaging utilizes different vibration properties and/or different excitation behavior between different types of tissue, in particular healthy tissue and diseased tissue or tumor tissue. These different types of behavior can be depicted in a magnetic resonance elastogram using magnetic resonance imaging.

Herein, in conjunction with magnetic resonance tomography, it is for example possible to capture the oscillation behavior of the patient's liver tissue by means of a magnetic resonance measurement and to derive the condition of the liver therefrom. However, magnetic resonance tomography places special demands on the elastography apparatus. For example, a drive unit, for example a motor for generating a drive torque for the elastography apparatus, needs to be arranged outside a scanner unit, e.g. outside a patient receiving region of the scanner unit. This in turn requires a long transmission shaft to transmit the drive torque to a vibration unit of the elastography apparatus.

### SUMMARY

The present disclosure provides a particularly compact space-saving elastography apparatus for magnetic resonance elastography examinations. The object is achieved by the various embodiments and features as discussed herein, including the claims.

The disclosure describes a magnetic resonance system comprising:
a magnetic resonance apparatus with a scanner unit having a main magnet, a gradient coil unit, and a radio-frequency antenna unit, and a patient receiving region at least partially surrounded by the scanner unit; and
an elastography apparatus embodied to excite regions of interest of a patient during a magnetic resonance elastography examination, comprising a vibration unit, a magnetic resonance-compatible drive unit, and a transmission unit for transmitting a drive torque generated by the magnetic resonance-compatible drive unit to the vibration unit.

According to the disclosure, the magnetic resonance-compatible drive unit has a magnetic resonance-compatible motor.

The magnetic resonance system with the magnetic resonance apparatus and the elastography apparatus may be configured to display different vibration properties and/or different excitation behavior of different tissue types, such as, for example, healthy tissue and tumor tissue or diseased tissue, in a magnetic resonance elastogram. For this purpose, the elastography apparatus may be used to excite a region of interest of the patient by means of vibrations and/or oscillations. The different oscillation behavior and/or vibration behavior of the different tissue types is displayed in a magnetic resonance elastogram by means of magnetic resonance imaging.

The magnetic resonance apparatus may comprise a medical and/or diagnostic magnetic resonance apparatus configured or embodied to capture medical and/or diagnostic image data, in particular medical and/or diagnostic magnetic resonance image data of a patient. The magnetic resonance apparatus comprises the scanner unit for this purpose. The scanner unit may comprise a magnet unit for capturing the medical and/or diagnostic image data. Herein, the scanner unit, e.g. the magnet unit, advantageously comprises a main magnet, a gradient coil unit and a radio-frequency antenna unit. The radio-frequency antenna unit is arranged within the scanner unit in a fixed manner and configured and/or embodied to emit an excitation pulse.

The main magnet is embodied to generate a homogeneous main magnetic field with a defined magnetic field strength, such as, for example, a magnetic field strength of 3 T or 1.5 T etc. The main magnet is e.g. embodied to generate a strong and constant main magnetic field. The homogeneous main magnetic field may be arranged and/or located within the patient receiving region of the magnetic resonance apparatus. The gradient coil unit is embodied to generate magnetic field gradients that are used for spatial encoding during imaging.

The patient receiving region is configured and/or embodied to receive the patient, e.g. the region of interest of the patient, for a medical magnetic resonance examination. The patient receiving region may comprise the region that is available to the patient during a magnetic resonance examination. For example, for this purpose, the patient receiving region is embodied as cylindrical in shape and/or is cylindrically surrounded by the scanner unit, e.g. the magnet unit, of the magnetic resonance apparatus. As an example, herein the scanner unit, e.g. the magnet unit, may comprise a housing that at least partially surrounds the patient receiving region. Herein, the housing may surround the patient receiving region in a cylindrical shape. Herein, the housing may be embodied in one piece with the radio-frequency antenna unit of the magnet unit and comprise a side of the radio-frequency antenna unit facing the patient receiving region.

A field of view (FoV) and/or an isocenter of the magnetic resonance apparatus may be arranged within the patient receiving region. The FoV may comprise a capture region of the magnetic resonance apparatus within which the conditions for capturing medical image data, e.g. magnetic resonance image data, are present within the patient receiving region, such as, for example, a homogeneous main magnetic field. The isocenter of the magnetic resonance apparatus may comprise the region and/or point within the magnetic resonance apparatus that has the optimal and/or ideal conditions for capturing medical image data. For instance, the isocenter comprises the most homogeneous magnetic field region within the magnetic resonance apparatus.

The vibration unit of the elastography apparatus is embodied to generate vibrations and/or pressure waves and to transmit these to the patient, e.g. to a region of interest of the patient. For this purpose, the vibration unit may be positioned close to the patient, e.g. close to the region of interest of the patient. Herein, the vibration unit is e.g. placed on the region of interest of the patient. To secure the position of the vibration unit, the vibration unit can also be attached and/or fixed to the patient with straps. The vibration unit is thus located within the patient receiving region during the examination.

A magnetic resonance-compatible drive unit, e.g. a magnetic resonance-compatible motor, is understood as a drive unit, e.g. a motor, for use with a magnetic resonance apparatus, wherein the drive unit, e.g. the motor, is not embodied for imaging. For example, herein, the magnetic resonance-compatible drive unit, e.g. the magnetic resonance-compatible motor, does not have any components embodied for imaging, thus advantageously enabling impairment of a magnetic resonance measurement to be avoided.

The transmission unit of the elastography apparatus may comprise a drive shaft for transmitting the drive torque, e.g. a rotational torque, from the magnetic resonance-compatible drive unit, e.g. the magnetic resonance-compatible motor, to the vibration unit. The transmission unit, e.g. the drive shaft, may be embodied as magnetic resonance-compatible and e.g. not embodied for imaging. The transmission unit, e.g. the drive shaft, may be embodied as flexible, in order, for example, to take into account different anatomies of different patients when arranging and/or positioning the elastography apparatus. Herein, the drive shaft may comprise a flexible shaft. Alternatively or additionally, the transmission unit, e.g. the drive shaft, may also have at least one joint, e.g. a cardan joint, or also be embodied as a cardan shaft. The transmission unit, e.g. the drive shaft, may also be encased, for example in a hose, thus advantageously preventing injury to the patient during the transmission of drive torque, e.g. a rotational torque, to the vibration unit.

The elastography apparatus can also comprise a coupling unit by means of which the elastography apparatus is coupled and/or connected to the magnetic resonance apparatus with regard to data exchange. For example, the coupling unit may be used to exchange control signals between the magnetic resonance apparatus and the elastography apparatus. Herein, the coupling unit can comprise a wired coupling unit. In addition, a cableless and/or wireless coupling unit is also possible at any time. The coupling unit may comprise a data transmission unit between a control unit of the elastography apparatus and a control unit of the magnetic resonance apparatus.

The disclosure can advantageously provide a particularly space-saving and compact elastography apparatus for magnetic resonance elastography examinations. Particularly advantageously, the magnetic resonance-compatible drive unit, e.g. the magnetic resonance-compatible motor, can be positioned particularly close to the region of interest of the patient and thus to the vibration unit, thus enabling a short transmission unit, in particular a short drive shaft, to be used to transmit the drive torque.

In an advantageous development of the magnetic resonance system, it can be provided that the magnetic resonance-compatible motor is arranged within a homogeneous main magnetic field generated by the main magnet, wherein the magnetic resonance-compatible motor comprises a stator and the stator comprises a dominant component of the main magnetic field. Herein, the magnetic resonance-compatible motor comprises an electromagnetic motor with a stator comprising the dominant component of the main magnetic field of the magnetic resonance apparatus. Within the patient receiving region and/or close to the isocenter, the main magnetic field of the main magnet comprises only a dominant component BO in the z-direction of the magnetic resonance apparatus. Herein, the z-direction of the magnetic resonance apparatus is aligned parallel to a longitudinal direction of the patient receiving region. The dominant component of the main magnetic field and/or a stray field may also be aligned in the z-direction of the magnetic resonance apparatus outside the FoV and/or outside the patient receiving region. This dominant component of the main magnetic field serves as a stator for the magnetic resonance-compatible drive unit, e.g. the electromagnetic magnetic resonance-compatible motor. Herein, the dominant component of the main magnetic field may be aligned perpendicular to a motor axis of the magnetic resonance-compatible drive unit.

The magnetic resonance-compatible motor may comprise a rotor and/or a rotatable motor element. The rotor and/or the rotatable motor element comprises at least one rotatably mounted coil element with a coil axis aligned perpendicular to the dominant component of the main magnetic field. The rotatably mounted motor element is embodied to generate a drive torque of the magnetic resonance-compatible motor. Herein, a rotary motion of the rotatably mounted motor element can also only comprise a partial rotation, and not a complete rotation, about the motor axis. The at least one rotatably mounted motor element is preferably rotatably mounted in both directions about the coil axis. The rotatably mounted motor element has at least one coil winding and preferably a plurality of coil windings, thus enabling a large force, e.g. a large Lorentz force, to act on the at least one rotatably mounted motor element to generate a drive torque. Particularly advantageously, the at least one rotatably mounted motor element, e.g. the rotatably mounted coil element, of the magnetic resonance-compatible motor has a copper wire coil with a plurality of coil windings. When the at least one rotatably mounted motor element rotates about the coil axis, the inclination of the coil surface with respect to the main magnetic field and/or the dominant component of the main magnetic field of the main magnet changes. Herein, the direction of rotation and/or rotational direction of the rotatably mounted coil element is dependent on the direction of a current flowing through the rotatably mounted coil element. Thus, a direction of rotation and/or rotational direction can also be changed by changing the direction of the current.

A further embodiment of the magnetic resonance-compatible motor, more precisely a stepper motor, corresponds to the explanations of the magnetic resonance-compatible stepper motor in the patent specification DE 10 2020 211 326 A1, to which explicit reference is hereby made.

This embodiment of the disclosure has the advantage that a drive torque of the magnetic resonance-compatible motor, and thus for generating vibrations and/or pressure waves for exciting a region of interest and/or tissue of the patient, can be generated particularly easily.

In an advantageous development of the magnetic resonance system, it can be provided that the main magnet has at least one magnetic coil for generating the homogeneous main magnetic field, wherein the magnetic resonance-compatible motor for generating the drive torque for the vibration unit is arranged within the patient receiving region in a region covered by the magnetic coil. The main magnet may e.g. comprise a Helmholtz coil pair with two magnetic coils. Herein, a first magnetic coil of the Helmholtz coil pair is arranged in a front region of the magnet unit, e.g. the main magnet, and a second magnetic coil of the Helmholtz coil pair is arranged in a rear region of the magnet unit, e.g. the main magnet. Herein, the front region extends 10 cm to 30 cm from an insertion opening of the patient receiving region into the patient receiving region and the magnet unit. Herein, the rear region extends 10 cm to 30 cm from an end opening of the patient receiving region into the patient receiving region and the magnet unit.

The FoV of the magnet unit with the homogeneous main magnetic field may e.g. be arranged between the two magnetic coils of the Helmholtz coil pair. In contrast, the magnetic field is maximized at the positions, e.g. in the z-direction, of the two magnetic coils of the Helmholtz coil pair. The magnetic resonance-compatible motor may be arranged in the z-direction at the same position as a magnetic coil of the Helmholtz coil pair, so that the magnetic resonance-compatible motor is arranged in a region covered by the magnetic coil. For easy positioning of the magnetic resonance-compatible motor within the patient receiving region and thus within the main magnetic field during a magnetic resonance elastography examination, the magnetic resonance-compatible motor is arranged at a position of the first magnetic coil of the Helmholtz coil pair in the vicinity of the entrance opening, thus enabling easy accessibility for a user. This embodiment of the disclosure has the advantage that the magnetic resonance-compatible motor can be arranged in a maximum magnetic field and thus the magnetic resonance-compatible motor can be operated with a low current intensity for generating a defined drive torque.

In an advantageous development of the magnetic resonance system, it can be provided that the magnetic resonance-compatible motor has a magnetic field sensor. The magnetic field sensor may be embodied to determine and/or capture a magnitude of the main magnetic field, e.g. a magnitude of the dominant component of the main magnetic field, at the position of the magnetic resonance-compatible motor. Herein, the magnetic field sensor may comprise a Hall sensor for capturing a magnitude of the main magnetic field. This embodiment of the disclosure has the advantage that an exact magnitude of the main magnetic field at the position of the motor is captured during a magnetic resonance elastography examination. Based on the exact magnitude of the main magnetic field, a defined drive torque, e.g. a rotational torque, can be generated and/or produced for the vibration unit by setting a corresponding current intensity for the magnetic resonance-compatible motor. A current intensity may be set on the magnetic resonance-compatible motor by means of a motor driver unit and a control unit of the elastography apparatus.

In an advantageous development of the magnetic resonance system, it can be provided that the elastography apparatus has a holding apparatus on which the magnetic resonance-compatible motor is arranged during a magnetic resonance elastography examination. The holding apparatus may have at least one attaching element, wherein the magnetic resonance-compatible motor can be attached to the holding apparatus by means of the attaching element. Advantageously, the holding apparatus is embodied such that the magnetic resonance-compatible motor is arranged in a defined position on the holding apparatus with respect to the dominant component of the main magnetic field, e.g. with a coil axis perpendicular to the dominant component of the main magnetic field, during a magnetic resonance elastography examination. In this way, it is advantageously possible to achieve secure and stable positioning of the magnetic resonance-compatible motor during a magnetic resonance elastography examination. For example, the magnetic resonance-compatible motor retains its position during a magnetic resonance elastography examination with respect to the dominant component of the main magnetic field, thus enabling the settings selected for generating and/or producing a defined drive torque for the vibration unit to be retained unchanged. In addition, it is advantageously possible to prevent slippage and/or incorrect positioning of the magnetic resonance-compatible motor, as can be the case, for example, when the magnetic resonance-compatible motor is placed on the patient during a magnetic resonance elastography examination.

In an advantageous development of the magnetic resonance system, it can be provided that the magnetic resonance apparatus has a patient support apparatus with a patient table that is movable within the patient receiving region, wherein the holding apparatus is embodied for detachable attachment to the patient table.

The magnetic resonance apparatus has the patient support apparatus for positioning the patient, e.g. the region of interest of the patient, within the patient receiving region. The patient support apparatus is embodied to support the patient. The patient support apparatus may have e.g. a movable patient table, which is embodied to be movable e.g. within the patient receiving region of the magnetic resonance apparatus. Herein, the patient table is embodied as movable in the longitudinal direction of the patient receiving region and/or in the z-direction within the patient receiving region. For a magnetic resonance elastography examination, the patient is first positioned on the patient table of the patient support apparatus and the elastography apparatus is arranged and/or positioned on the patient and/or on the patient table. In addition, further additional units can also be positioned on the patient, such as, for example, an injection unit and/or an ECG unit and/or a positioning pad, etc. The patient table then moves together with the patient into the patient receiving region until the region of interest of the patient is arranged within the isocenter of the magnetic resonance apparatus. The holding apparatus may be embodied for detachable attachment to the patient table, thus enabling easy removal of the holding apparatus and hence also of the magnetic resonance-compatible motor. This embodiment of the disclosure has the advantage that a simple and secure arrangement of the elastography apparatus, e.g. the magnetic resonance-compatible motor for a magnetic resonance elastography examination, can be achieved. In this way, the holding apparatus can be securely arranged on and/or fastened to the patient table for a magnetic resonance elastography examination.

In an advantageous development of the magnetic resonance system, it can be provided that the holding apparatus comprises a convex holding arc with two end regions and a central attachment region, wherein the two end regions are embodied for detachable attachment to the patient table and the central attachment region is embodied for an arrangement of the magnetic resonance-compatible motor. The patient table may have two attaching rails, which in each case extend in the longitudinal direction of the patient table. Herein, the two attaching rails are in each case arranged on a lateral edge region of the patient table, wherein a supporting surface and/or a supporting region of the patient table for supporting the patient is arranged between the two lateral edge regions, and thus the two attaching rails of the patient table. The two attaching rails can be used to securely attach and/or arrange accessory units that are required for an upcoming magnetic resonance examination and/or an upcoming magnetic resonance elastography examination on the patient table.

The two end regions of the convex holding arc are embodied for arrangement on and/or attachment to the two attaching rails of the patient table, wherein a first end region is arranged in a first of the two attaching rails and a second end region is arranged in a second of the two attaching rails. In an attached position on the patient table, the convex holding arc curves over the supporting surface and/or the supporting region of the patient table. Herein, the convex holding arc curves from a first side of the patient table to a second side of the patient table, wherein the supporting surface of the patient table is arranged between the two sides. This means that the convex holding arc also curves over the patient arranged on the patient table, for example over a leg region of the patient. In a top view of the patient table from above, the convex holding arc is embodied as convex in a position arranged on the patient table. The attachment region for attaching and/or arranging the magnetic resonance-compatible motor is arranged in the central region of the convex holding arc. The central attachment region of the convex holding arc may have a preferred attachment position for the magnetic resonance-compatible motor, so that the magnetic resonance-compatible motor is in a position in which a motor axis of the magnetic resonance-compatible motor is aligned perpendicular to the dominant component of the main magnetic field.

This embodiment of the disclosure enables a secure and stable arrangement of the magnetic resonance-compatible stepper motor. In addition, this type of arrangement of the magnetic resonance-compatible motor advantageously prevents adverse effects on the patient during positioning on the patient table.

In an advantageous development of the magnetic resonance system, it can be provided that the magnetic resonance apparatus has a positioning pad for positioning a patient, wherein the magnetic resonance-compatible motor is arranged within the positioning pad. The positioning pad may be embodied for positioning and/or comfortably supporting partial regions of the patient, such as, for example, a positioning pad for positioning and/or supporting the patient's legs or knees. For example, such a positioning pad can have a receiving region and/or a pocket embodied to receive the magnetic resonance-compatible motor. The receiving region and/or the pocket of the positioning pad may have a preferred attachment position for the magnetic resonance-compatible motor, so that the magnetic resonance-compatible motor is in a position in which a motor axis of the magnetic resonance-compatible motor is aligned perpendicular to the dominant component of the main magnetic field. This embodiment of the disclosure enables a secure and protected arrangement of the magnetic resonance-compatible motor during a magnetic resonance elastography examination. In addition, this type of arrangement of the magnetic resonance-compatible motor advantageously prevents adverse effects on the patient during positioning on the patient table.

In an advantageous development of the magnetic resonance system, it can be provided that the transmission unit has a variable-length drive shaft. For instance, the length of the drive shaft can be set by a user during positioning of the patient and the elastography apparatus on the patient table. This enables simple positioning of the elastography apparatus on the patient, since the magnetic resonance-compatible drive unit, e.g. the magnetic resonance-compatible motor, can be positioned on the patient and/or on the patient table independently of the length of the transmission unit, e.g. the drive shaft. This may also enable the length of the drive shaft to be adapted to the patient's height.

In order to prevent any impairment of magnetic resonance data capturing during a magnetic resonance elastography examination, the magnetic resonance-compatible motor should be at a minimum distance from the region of interest of the patient. Depending upon the field strength of the main magnet of the magnetic resonance apparatus, herein the minimum distance may be within any suitable range of distances, such as between 4 cm and 90 cm for instance. The variable-length drive shaft can be used to maintain this minimum distance between the region of interest and the magnetic resonance-compatible motor and also to provide a compact elastography apparatus.

Herein, the variable-length drive shaft can be embodied as telescopic. For example, the variable-length drive shaft can comprise two or more interlocking rods, for example rods with a square or hexagonal cross section. Herein, the interlocking rods may have different cross sections.

In an advantageous development of the magnetic resonance system, it can be provided that the vibration unit has a vibration element, wherein the drive torque generated by the magnetic resonance-compatible motor can be transmitted to the vibration element. The vibration element may comprise an oscillating mass and/or a vibrating mass embodied to generate oscillation and/or vibration. In addition, the vibration element is embodied to transmit the generated oscillation and/or vibration to the patient, e.g. to the region of interest of the patient. Reversing the polarity of a current flowing through the magnetic resonance-compatible motor enables the generation of a drive torque, e.g. a rotational torque, that changes direction. In this way, a back-and-forth oscillating rotational torque, which can be transmitted directly to the vibration element, e.g. the oscillating mass, can be generated. This also enables a particularly compact design of the vibration unit, since additional vibration-generating elements can advantageously be dispensed with.

Alternatively, the vibration unit may also have an eccentric element, wherein the drive torque generated by the magnetic resonance-compatible motor can be transmitted to the eccentric element. In addition, the vibration unit may comprise a locking pawl arranged upstream of the eccentric element within the vibration unit, so that a rotational torque is always transmitted in the same direction to the eccentric element in order to generate vibration and/or oscillation.

In an advantageous development of the magnetic resonance system, it can be provided that the elastography apparatus comprises a motor driver unit and a shield housing, wherein the motor driver unit is arranged in the shield housing. The motor driver unit e.g. comprises a circuit and/or a circuit unit for actuating the magnetic resonance-compatible stepper motor. The motor driver unit may comprise an H-bridge for voltage regulation of the magnetic resonance-compatible stepper motor. The shield housing shields the motor driver unit from the magnet unit. As an example, the shield housing may shield the motor driver unit from the magnet unit with respect to radio-frequency radiation. The shield housing advantageously comprises an electromagnetic filter element, wherein the electromagnetic filter element filters all outgoing signals from the motor driver unit in order to avoid interactions of the magnet unit.

Due to the arrangement of the motor driver unit in the shield housing, the motor driver unit can also be arranged within the patient receiving region and/or in a region in which a stray field of the main magnetic field is present. This advantageously enables undesirable interaction between the motor driver unit and the scanner unit to be reduced and/or prevented.

In an advantageous development of the magnetic resonance system, it can be provided that the elastography apparatus has a control unit, wherein the control unit is embodied to synchronize the elastography apparatus with a measurement sequence of the magnetic resonance elastography examination.

The control unit may e.g. be arranged outside the patient receiving region of the scanner unit. In addition, the control unit may have a data connection to the magnetic resonance apparatus, e.g. a magnetic resonance control unit of the magnetic resonance apparatus. Herein, the data connection can be wired or wireless.

The control unit of the elastography apparatus comprises at least one computing module and/or a processor. The control unit may e.g. be embodied to execute computer-readable instructions. For example, the control unit may comprise a storage unit, wherein computer-readable information is stored on the storage unit, wherein the control unit is embodied to load the computer-readable information from the storage unit and execute the computer-readable information. The components of the control unit can be predominantly embodied in the form of software components. However, in principle, e.g. when particularly fast calculations are involved, these components can also to some extent be realized in the form of software-supported hardware components, for example FPGAs or the like. Likewise, the required interfaces can be embodied as software interfaces, for example if only the transfer of data from other software components is involved. However, they can also be embodied as hardware interfaces that are actuated by suitable software. Of course, it is also conceivable for a plurality of said components to be combined in the form of a single software component or software-supported hardware component.

In an operating mode of the elastography apparatus, the control unit sends control signals directly to the motor driver unit and thus controls the elastography apparatus. The control unit may control the motor driver unit in such a way that an oscillation process and/or excitation process is synchronized with the measurement sequence of the magnetic resonance elastography examination to be played out. This can provide an advantageous coordination between the magnetic resonance apparatus and the elastography apparatus. Herein, preferably, the start and end of the measurement sequence are coordinated with the oscillation process and/or excitation process of the elastography apparatus. For synchronization with the measurement sequence, the control unit of the elastography apparatus can also provide at least one trigger signal of a magnetic resonance control unit (also referred to as a magnetic resonance sequence control unit), so that the measurement sequence is triggered by the elastography apparatus. Herein, a settling process can also be provided for the elastography apparatus until a mechanical system, e.g. the magnetic resonance-compatible motor and the vibration unit, has settled to an excitation frequency. This settling process is taken into account by the control unit when providing the trigger signal. An excitation process and/or an oscillation process for excitation by means of the elastography apparatus may comprise any suitable frequency, e.g. frequencies between 50 Hz and 1000 Hz. An excitation frequency and/or oscillation frequency may be, for instance, approximately 100 Hz.

A measurement sequence may comprise a magnetic resonance sequence, wherein a magnetic resonance sequence may comprise a temporal sequence of radio-frequency pulses. For example, a magnetic resonance sequence can comprise a T1-weighted sequence or a T2-weighted sequence or a spin-echo sequence, etc. Herein, the individual magnetic resonance sequences differ in terms of their sequence parameters.

In an advantageous development of the magnetic resonance system, it can be provided that the elastography apparatus comprises an optical transmission unit arranged between the motor driver unit and the control unit. The optical connection unit may comprise optical conductors, for example fiber-optic conductors and/or fiber-optic cables. This enables interference-free signal transmission between the motor driver unit and the control unit.

### BRIEF DESCRIPTION OF THE DRAWINGS

Further advantages, features and details of the disclosure will emerge from the exemplary embodiments described below and from the drawings.

The drawings show:

FIG. 1 illustrates a magnetic resonance system according to the disclosure with an example magnetic resonance apparatus and an example elastography apparatus in a schematic representation;

FIG. 2 illustrates positioning of an example magnetic resonance-compatible drive unit of an elastography apparatus with respect to a main magnet of the magnetic resonance apparatus;

FIG. 3 illustrates an example structure of the elastography apparatus;

FIG. 4 illustrates a sectional view of the example structure of the magnetic resonance-compatible motor;

FIG. 5 illustrates a first exemplary embodiment of the elastography apparatus with an example holding apparatus;

FIG. 6 illustrates a second exemplary embodiment of the elastography apparatus with an example arrangement of the magnetic resonance-compatible drive unit in a positioning pad; and

FIG. 7 illustrates a side view of the second exemplary embodiment.

### DETAILED DESCRIPTION OF THE DISCLOSURE

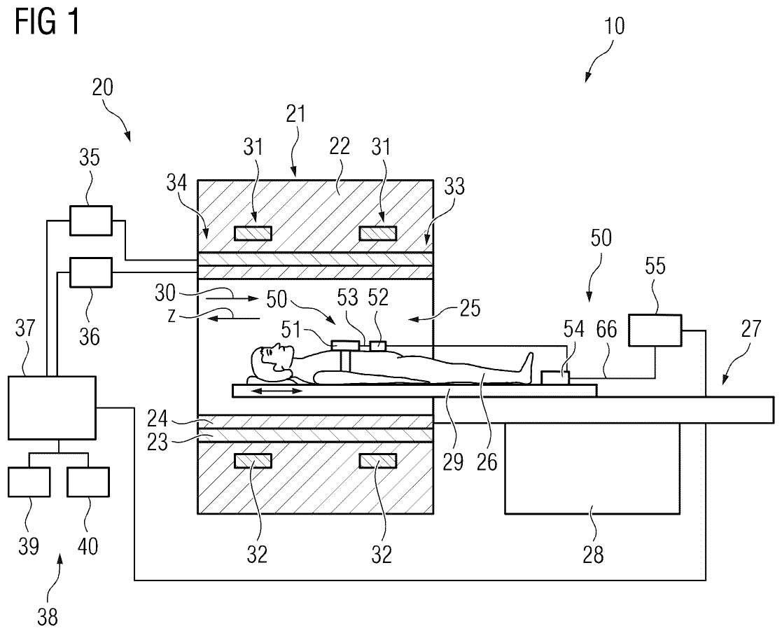

FIG. 1 is a schematic representation of a magnetic resonance system 10 with a magnetic resonance apparatus 20 and an elastography apparatus 50. The magnetic resonance apparatus 20 comprises a scanner unit (also referred to herein as a scanner) embodied as a magnet unit 21. The scanner unit, e.g. the magnet unit 21, comprises a main magnet 22, a gradient coil unit 23 (also referred to herein as gradient coils), and a radio-frequency antenna unit 24 (also referred to herein as RF circuitry). In addition, the magnetic resonance apparatus 20 has a patient receiving region 25 for receiving a patient 26 for a magnetic resonance examination and/or a magnetic resonance elastography examination. In the present exemplary embodiment, the patient receiving region 25 is cylindrical in shape and is surrounded in a circumferential direction by the scanner unit, e.g. by the magnet unit 21. However, in principle, different embodiments of the patient receiving region 25 are conceivable.

For positioning the patient 26, e.g. a region of interest of the patient 26, within the patient receiving region 25, the magnetic resonance apparatus 20 has a patient support apparatus 27. The patient support apparatus 27 has a base unit 28 and a patient table 29 that is movable with respect to the base unit 28. For positioning the patient 26, e.g. the region of interest of the patient 26, the patient table 29 is embodied as movable within the patient receiving region 25. Herein, in the patient table 29 is mounted so as to be movable in the direction of a longitudinal extension of the patient receiving region 25 and/or in the z-direction.

The main magnet 22 of the magnet unit 21 is embodied to generate a strong and in particular constant main magnetic field 30. The main magnet 22 has a Helmholtz coil pair 31 with two magnetic coils 32 for generating the homogeneous main magnetic field 30. Herein, a first magnetic coil 32 of the Helmholtz coil pair 31 is arranged in a front region 33 of the magnet unit 21, e.g. the main magnet 22, and a second magnetic coil 32 of the Helmholtz coil pair 31 is arranged in a rear region 34 of the magnet unit 21, e.g. the main magnet 22. Herein, the front region 33 extends 10 cm to 30 cm from an insertion opening of the patient receiving region 25 into the patient receiving region 25 and the magnet unit 21. Herein, the rear region 34 extends 10 cm to 30 cm from an end opening of the patient receiving region 25 into the patient receiving region 25 and the magnet unit 21. The FoV of the scanner unit with the homogeneous main magnetic field 30 mat be e.g. arranged between the two magnetic coils 32 of the Helmholtz coil pair 31.

The gradient coil unit 23 of the magnet unit 21 is embodied to generate magnetic field gradients that are used for spatial encoding during imaging. The gradient coil unit 23 is controlled by means of a gradient control unit 35 of the magnetic resonance apparatus 20. The radio-frequency antenna unit 24 of the magnet unit 21 is embodied to excite polarization that is established in the main magnetic field 30 generated by the main magnet 22. The radio-frequency antenna unit 24 is controlled by a radio-frequency antenna control unit 36 of the magnetic resonance apparatus 20 and radiates radio-frequency magnetic resonance sequences into the patient receiving region 25 of the magnetic resonance apparatus 20.

The magnetic resonance apparatus 20 has a magnetic resonance control unit 37 to control the main magnet 22, the gradient control unit 35, and to control the radio-frequency antenna control unit 36. The magnetic resonance control unit 37 centrally controls the magnetic resonance apparatus 20, such as, for example, performing a predetermined imaging gradient echo sequence. In addition, the magnetic resonance control unit 37 comprises an evaluation unit, not shown in detail, for evaluating medical image data captured during the magnetic resonance examination.

Moreover, the magnetic resonance apparatus 20 comprises a user interface 38, which is connected to the magnetic resonance control unit 37. Control information, such as, for example, imaging parameters, and reconstructed magnetic resonance images can be displayed on a display unit 39, for example on at least one monitor, of the user interface 38 for a medical operator. Furthermore, the user interface 38 has an input unit 40 by means of which information and/or parameters can be entered by a medical operator during a measurement process.

The magnetic resonance apparatus 20 depicted can of course comprise further components that magnetic resonance devices usually have. In addition, the general mode of operation of a magnetic resonance apparatus 20 is known to the person skilled in the art and so no detailed description of the further components will be given.

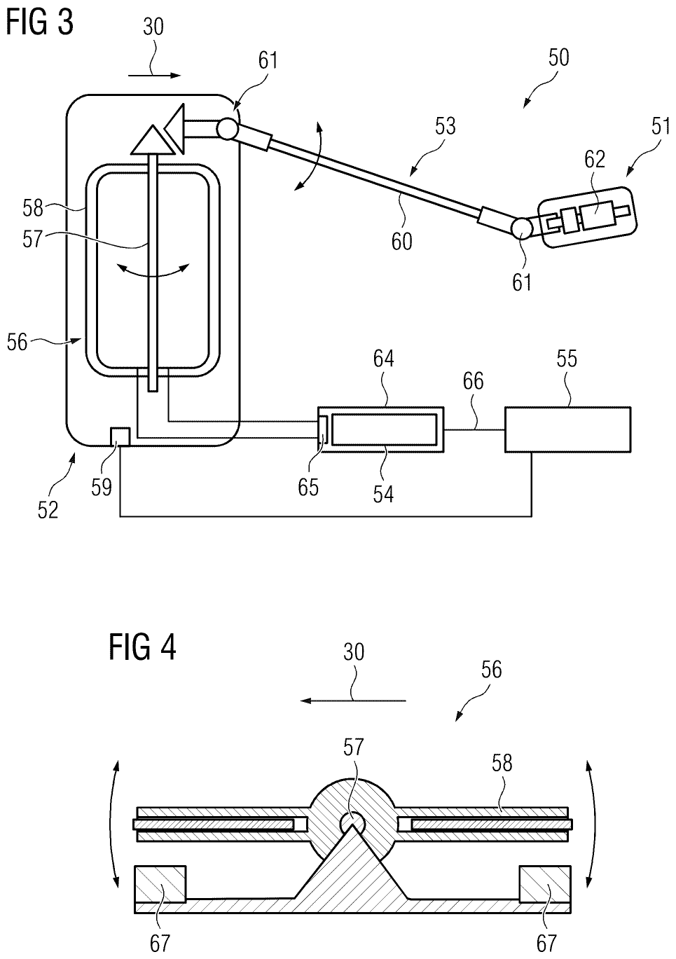

The elastography apparatus 50 of the magnetic resonance system 10 is embodied to excite a region of interest of the patient 26 during a magnetic resonance elastography examination. For this purpose, the elastography apparatus 50 comprises a vibration unit 51 (also referred to herein as a vibrator), a magnetic resonance-compatible drive unit 52 (also referred to herein as a magnetic resonance-compatible driver), and a transmission unit 53 (also referred to herein as a transmitter, a torque transmitter, or a vibration transmitter). In addition, the elastography apparatus 50 has a motor driver unit 54 (also referred to herein as a motor driver) and a control unit 55 (also referred to herein as a controller) (See FIG. 1 and FIG. 3).

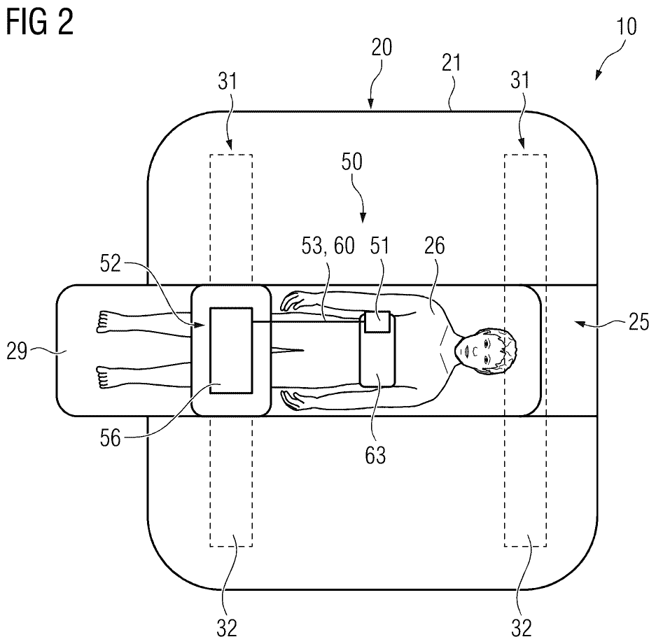

The magnetic resonance-compatible drive unit 52 is embodied to produce and/or generate a drive torque for the vibration unit 51 and has a magnetic resonance-compatible motor 56 for this purpose. Herein, the magnetic resonance-compatible motor 56 is arranged within the main magnetic field 30 generated by the main magnet 22. In addition, the magnetic resonance-compatible motor 56 comprises a stator, wherein the stator comprises a dominant component of the main magnetic field 30 of the main magnet 22. Within the patient receiving region 25 and/or close to the isocenter, the main magnetic field 30 of the main magnet 22 comprises only a dominant component BO in the z-direction of the magnetic resonance apparatus 20 (FIG. 1 and FIG. 2). Even outside the FoV and/or outside the patient receiving region 25, the dominant component of the main magnetic field 30 and/or of a stray field may be aligned in the z-direction of the magnetic resonance apparatus 20. This dominant component of the main magnetic field 30 serves as a stator for the magnetic resonance-compatible drive unit 52, e.g. the electromagnetic and magnetic resonance-compatible motor 56. Herein, the dominant component of the main magnetic field 30 is aligned perpendicular to a motor axis 57 of the magnetic resonance-compatible motor 56 (FIG. 3 and FIG. 4).

In addition, the magnetic resonance-compatible motor 56 comprises a rotor and/or a rotatable motor element 58 (FIG. 3). The rotor, and/or the rotatable motor element 58, comprises at least one rotatably mounted coil element with a coil axis aligned perpendicular to the dominant component of the main magnetic field 30 and formed by the motor axis 57. Herein, the at least one rotatably mounted motor element 58, e.g. the rotatably mounted coil element, of the magnetic resonance-compatible motor 56 has a copper wire coil with a plurality of coil windings. The rotatably mounted motor element 58 is embodied to generate a drive torque of the magnetic resonance-compatible motor 56. Herein, a rotary motion of the rotatably mounted motor element 58 can also comprise only a partial rotation, and not a complete rotation, about the motor axis 57. The at least one rotatably mounted motor element 58 may e.g. be rotatably mounted in both directions about the motor axis 57 (FIG. 4). Herein, a Lorentz force acts on the rotatably mounted motor element 58, which causes a rotation and thus a generation of the drive torque. Herein, when the at least one rotatably mounted motor element 58 rotates about the motor axis 57, e.g. the coil axis, the inclination of the coil surface with respect to the main magnetic field 30 and/or the dominant component of the main magnetic field 30 changes. Herein, a direction of rotation and/or a rotational direction of the rotatably mounted motor element 58 is dependent on the direction of a current flowing through the rotatably mounted motor element 58 (FIG. 3 and FIG. 4). To limit rotation in one direction, the magnetic resonance-compatible motor 56 has two stop elements 67, wherein a first stop element 67 limits the rotary motion of the rotatably mounted motor element 58 in a first direction of rotation and a second stop element 67 limits the rotary motion of the rotatably mounted motor element 58 in a first direction of rotation.

In order to utilize a maximum field strength of the main magnetic field 30, e.g. the dominant component of the main magnetic field 30, the magnetic resonance-compatible drive unit 52, e.g. the magnetic resonance-compatible motor 56, is arranged within the patient receiving region 25 in a region covered by a magnetic coil 32 of the Helmholtz coil pair 31 of the main magnet 22. Advantageously, the magnetic resonance-compatible drive unit 52, e.g. the magnetic resonance-compatible motor 56, is arranged at the same position in the z-direction as a magnetic coil 32 of the Helmholtz coil pair 31 of the main magnet 22, as can be seen in FIG. 2 of a schematic arrangement of the vibration unit 51 and the magnetic resonance-compatible motor 56 on the patient 26 within the patient receiving region 25. If, for example, the homogeneous main magnetic field 30 generated in the FoV by the Helmholtz coil pair 31, e.g. between the two magnetic coils 32 of the Helmholtz coil pair 31, has a magnetic field strength of 3.0 T, the maximum magnetic field strength at the position of one of the two magnetic coils 32 of the Helmholtz coil pair 31 is 3.6 T.

In the present exemplary embodiment, the magnetic resonance-compatible motor 56 additionally has a magnetic field sensor 59, for example a Hall sensor, in order to capture a magnetic field strength at the position of the magnetic resonance-compatible motor 56. However, such a magnetic field sensor 59 is optional and not absolutely necessary, provided that the magnetic resonance-compatible motor 56 is positioned at a position with a known magnetic field strength of the main magnetic field 30, e.g. the dominant component of the main magnetic field 30, during a magnetic resonance elastography examination.

The transmission unit 53 is embodied to transmit the drive torque generated by the magnetic resonance-compatible drive unit 52, e.g. the magnetic resonance-compatible motor 56, to the vibration unit 51. For this purpose, the transmission unit 53 has a drive shaft 60. Herein, the drive shaft 60 is embodied with a variable length. Herein, the variable-length drive shaft 60 can be embodied as telescopic. For example, the variable-length drive shaft 60 can comprise two or more interlocking rods, for example rods with a square or hexagonal cross section. Herein, the interlocking rods may e.g. have different cross sections. In addition, in FIG. 3, the drive shaft 60 has two joints 61, for example cardan joints, wherein, in FIG. 3, the drive shaft 60 is depicted with two joints 61 by way of example. Herein, an embodiment of the drive shaft 60 with more than two joints 61 is possible at any time. As an alternative to an embodiment of the drive shaft 60 with joints 61, the drive shaft 60 can also be embodied as flexible.

The vibration unit 51 has a vibration element 62 embodied to generate vibrations and/or oscillations and to transmit these vibrations and/or oscillations to the patient 26. For this purpose, the vibration unit 51, e.g. the vibration element 62, is positioned on the patient 26 in the region of interest of the patient 26. Herein, in addition, the vibration unit 51 can have an attaching strap 63, wherein the attaching strap 63 can be used to attach the vibration unit 51, e.g. the vibration element 62, to the region of interest of the patient 26. The vibration element 62 may comprise an oscillating mass and/or vibrating mass embodied to generate oscillation and/or vibration. In addition, the vibration element 62 is embodied to transmit the generated oscillation and/or vibration to the patient 26, e.g. to the region of interest.

Herein, the drive torque generated by the magnetic resonance-compatible drive unit 52, e.g. generated by the magnetic resonance-compatible motor 56, can be transmitted directly to the vibration element 62 by means of the transmission unit 53, e.g. the drive shaft 60 (FIG. 2 and FIG. 3). In addition, it can also be the case that the vibration element 62 is embodied as an eccentric element, wherein the drive torque generated by the magnetic resonance-compatible motor 56 can be transmitted to the eccentric element by means of the transmission unit 53, e.g. the drive shaft 60. Herein, in addition, the vibration unit 51 can comprise a locking pawl arranged upstream of the eccentric element, so that a rotational torque is always transmitted in the same direction to the eccentric element in order to generate vibration and/or oscillation.

The motor driver unit 54 of the elastography apparatus 50 may be arranged in a shield housing 64 of the elastography apparatus 50 (FIG. 3). The shield housing 64 shields the motor driver unit 54 from the magnet unit with respect to radio-frequency radiation. Advantageously, the shield housing 64 comprises an electromagnetic filter element 65, wherein the electromagnetic filter element 65 filters all outgoing signals from the motor driver unit 54 with respect to RF. The motor driver unit 54 e.g. comprises a circuit and/or a circuit unit for actuating the magnetic resonance-compatible motor 56. The motor driver unit 54 may e.g. comprise an H-bridge for voltage regulation of the magnetic resonance-compatible motor 56. Due to the arrangement of the motor driver unit 54 within the shield housing 64, the motor driver unit 54 can be arranged both within the patient receiving region 25 and outside the patient receiving region 25. In the present exemplary embodiment, the motor driver unit 54 is arranged within the patient receiving region 25 and/or in a stray field region of the main magnetic field 30 (FIG. 2).

To actuate the magnetic resonance-compatible motor 56, a defined current is provided for the magnetic resonance-compatible motor 56 by the motor driver unit 54, e.g. the H-bridge, and transmitted directly to the magnetic resonance-compatible motor 56. Herein, such currents for operating the magnetic resonance-compatible motor 56, and thus for generating a drive torque for the vibration unit, can have a current intensity of between 0.5 A and maximum 10 A.

The control unit 55 of the elastography apparatus 50 is embodied to control the elastography apparatus 50. In an operating mode of the elastography apparatus 50, the control unit 55 sends control signals directly to the motor driver unit 54 and thus controls the elastography apparatus 50. To transmit control signals between the control unit 55 and the motor driver unit 54, the elastography apparatus 50 has an optical connection unit 66 (FIG. 3). The optical connection unit 66 may comprises optical conductors, for example fiber-optic conductors and/or fiber-optic cables. As an alternative to an optical connection unit 66, the connection unit 66 can also be galvanic components or be wireless.

In addition, the control unit 55 is embodied to synchronize the elastography apparatus 50 with a measurement sequence of the magnetic resonance apparatus 20 during a magnetic resonance elastography examination. The control unit 55 may control the motor driver unit 54 such that an oscillation process and/or excitation process is synchronized with the measurement sequence of the magnetic resonance apparatus 20 to be played out during a magnetic resonance elastography examination.

Herein, a start and an end of the measurement sequence are synchronized with the oscillation process and/or excitation process. For synchronization with the measurement sequence, the control unit 55 of the elastography apparatus 50 can also provide at least one trigger signal of the magnetic resonance control unit 37, so that the measurement sequence is started triggered by the elastography apparatus 50. Herein, a settling process can also be provided for the elastography apparatus 50 until a mechanical system, e.g. the magnetic resonance-compatible motor 56 and the vibration unit 51, has settled to an excitation frequency. This settling process is taken into account by the control unit 55 when providing the trigger signal. An excitation process and/or an oscillation process for excitation by means of the elastography apparatus 50 may comprise any suitable frequency, e.g. a frequency between 50 Hz and 1000 Hz. An excitation frequency and/or an oscillation frequency may ebb for instance approximately 100 Hz.

If the elastography apparatus 50, e.g. the magnetic resonance-compatible motor 56, has a magnetic field sensor 59, the data captured by the magnetic field sensor 59 is transmitted to the control unit 55. On the basis of the captured magnetic field strength, a current to flow through the magnetic resonance-compatible motor 56 can be ascertained by the control unit 55 and set by the motor driver unit 54 in order to obtain an advantageous drive torque for the vibration unit 51.

The elastography apparatus 50 depicted can of course comprise further components that elastography devices usually have. In addition, the general mode of operation of an elastography apparatus 50 is known to the person skilled in the art and so no detailed description of the further components will be given.

FIG. 5 depicts a first exemplary embodiment for an arrangement and/or positioning of the elastography apparatus 50 for a magnetic resonance elastography examination. Substantially identical components, features, and functions of the magnetic resonance system 10, e.g. the magnetic resonance apparatus 20 and the elastography apparatus 50, are generally given the same reference symbols. The following description is substantially limited to the differences from the exemplary embodiment in FIG. 1 to FIG. 4, wherein reference is made to the description of the exemplary embodiment in FIG. 1 to FIG. 4 with regard to components, features and functions that remain the same.

An embodiment of the magnetic resonance-compatible drive unit 52, the vibration unit 51, the transmission unit 53, the motor driver unit 54, and the control unit 55 of the elastography apparatus 50 corresponds to the explanations for FIG. 1 to FIG. 4, to which reference is hereby made.

For a secure and stable arrangement and/or positioning of the magnetic resonance-compatible drive unit 52, e.g. the magnetic resonance-compatible motor 56, in the present exemplary embodiment, the elastography apparatus 50 has a holding apparatus 70. The holding apparatus 70 is embodied for detachable arrangement and/or positioning on the patient table 29 of the patient support apparatus 27.

The patient table 29 has two attaching rails 41, which in each case extend in the longitudinal direction of the patient table 29. Herein, the two attaching rails 41 are in each case arranged on a lateral edge region 42 of the patient table 29, wherein a supporting surface 43 and/or a supporting region of the patient table 29 for supporting the patient 26 is arranged between the two lateral edge regions 42 and thus the two attaching rails 41 of the patient table 29. The two attaching rails 41 can be used to securely attach and/or arrange accessory units that are required for an upcoming magnetic resonance examination and/or an upcoming magnetic resonance elastography examination on the patient table 29.

FIG. 5 shows a section through the patient table 29 with the holding apparatus 70 arranged on the patient table 29. To attach the holding apparatus 70 on the two attaching rails 41 of the patient table 29, the holding apparatus 70 is embodied as a convex holding arc. Herein, this convex holding arc comprises two end regions 71, wherein the two end regions 71 are arranged on opposite sides in the longitudinal extension of the convex holding arc. In each case, the two end regions 71 comprise an attaching element 72 for attaching the convex holding arc to the respective attaching rails 41. In addition, the convex holding arc comprises a central attachment region 73, wherein the central attachment region 73 is embodied for arranging and/or attaching the magnetic resonance-compatible motor 56. If the convex holding arc is arranged on the patient table 29, the convex holding arc curves over the supporting surface 43 and the supporting region of the patient table 29. Thus, the convex holding arc also curves over the patient 26 positioned on the patient table 29.

The convex holding arc may e.g. be positioned on the patient table 29 such that, in an examination position of the patient table 29, the convex holding arc, and thus the magnetic resonance-compatible motor 56 attached to the convex holding arc, is positioned at a z-position within the patient receiving region 25 on which a magnetic coil 32 of the Helmholtz coil pair 31 of the main magnet 22 is also arranged. In addition, e.g. the attachment region 73 is embodied such that a motor axis 57, e.g. an axis of rotation, of the magnetic resonance-compatible motor 56 is aligned perpendicular to the dominant component of the main magnetic field 30 when the magnetic resonance-compatible motor 56 is positioned on the convex holding arc and the convex holding arc is positioned on the patient table 29.

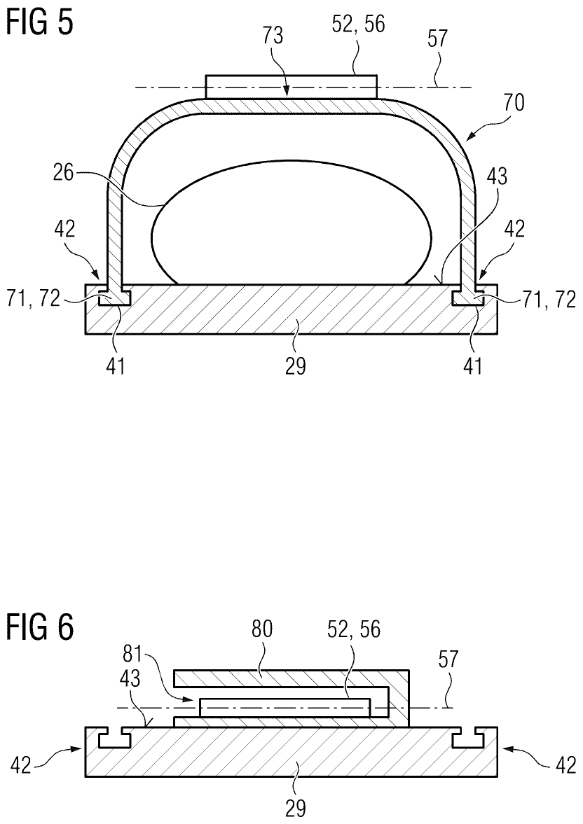

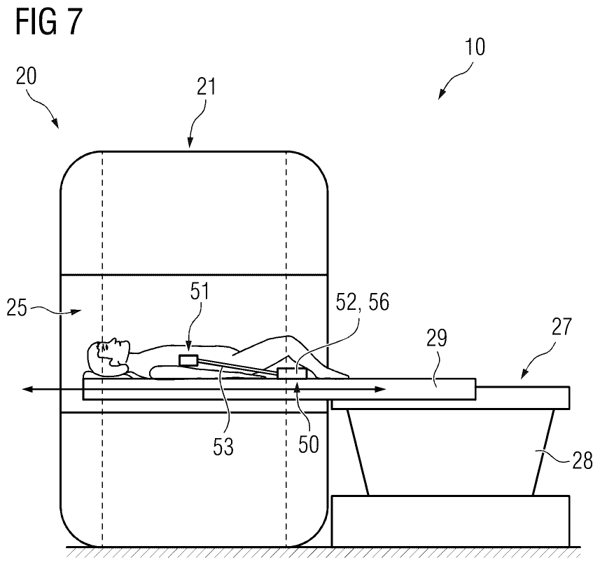

FIG. 6 and FIG. 7 show a second exemplary embodiment for an arrangement and/or positioning of the elastography apparatus 50 for a magnetic resonance elastography examination. Substantially identical components, features and functions of the magnetic resonance system 10, e.g. the magnetic resonance apparatus 20 and the elastography apparatus 50, are generally given the same reference symbols. The following description is substantially limited to the difference from the exemplary embodiment in FIG. 1 to FIG. 4, wherein reference is made to the description of the exemplary embodiment in FIG. 1 to FIG. 4 with regard to components, features and functions that remain the same.

An embodiment of the magnetic resonance-compatible drive unit 52, the vibration unit 51, the transmission unit 53, the motor driver unit 54 and the control unit 55 of the elastography apparatus 50 correspond to the explanations for FIG. 1 to FIG. 4, to which reference is hereby made.

The magnetic resonance apparatus 20 in FIGS. 6 and 7 comprises a positioning pad 80 embodied to support and/or position the patient 26 during a magnetic resonance elastography examination. For example, such a positioning pad 80 can be used for supporting and/or positioning knees, e.g. used as a pad under the knees of the patient 26. Herein, the positioning pad 80 has a pocket and/or a receiving region 81 embodied to receive the magnetic resonance-compatible motor 56 of the elastography apparatus 50. The pocket and/or the receiving region 81 is embodied such that a motor axis 57, e.g. an axis of rotation, of the magnetic resonance-compatible motor 56 is aligned perpendicular to the dominant component of the main magnetic field 30 when the magnetic resonance-compatible motor 56 is positioned within the pocket and/or the receiving region 81 of the positioning pad 80 and the positioning pad is positioned on the patient table 29 (FIG. 7). FIG. 6 shows a section through the patient table 29 with the positioning pad 80 arranged on the patient table 29. FIG. 7 shows a side view of the patient table 29 with a patient 26 positioned on the patient table 29 and an elastography apparatus 50. In addition to the magnetic resonance-compatible motor 56 positioned and/or arranged within the positioning pad 80, FIG. 7 also shows the transmission unit 53 and the vibration unit 51, which is positioned on the region of interest of the patient 26.

Although the disclosure has been described in greater detail by the preferred exemplary embodiment, the disclosure is not restricted by the disclosed examples and other variations can be derived herefrom by the person skilled in the art without departing from the scope of protection of the disclosure.

Independent of the grammatical term usage, individuals with male, female or other gender identities are included within the term.

The various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “circuitry,” “controllers,” “processors,” or “processing circuitry,” or alternatively as noted herein.

What is claimed is:

1. A magnetic resonance system, comprising:
a scanner including a main magnet;
a patient receiving region at least partially surrounded by the scanner; and
an elastography apparatus configured to excite regions of interest of a patient in the patient receiving region during a magnetic resonance elastography examination, the elastography apparatus comprising:
a vibrator;
a magnetic resonance-compatible driver comprising a magnetic resonance-compatible motor; and
a torque transmitter configured to transmit a drive torque generated by the magnetic resonance-compatible driver to the vibrator.

2. The magnetic resonance system as claimed in claim 1, wherein the magnetic resonance-compatible motor is arranged within a homogeneous main magnetic field generated by the main magnet, and comprises a stator comprising a dominant component of the homogeneous main magnetic field of the main magnet.

3. The magnetic resonance system as claimed in claim 1, wherein the main magnet includes a magnetic coil configured to generate a homogeneous main magnetic field, and
wherein the magnetic resonance-compatible motor is arranged within the patient receiving region in a region covered by the magnetic coil.

4. The magnetic resonance system as claimed in claim 1, wherein the magnetic resonance-compatible motor includes a magnetic field sensor.

5. The magnetic resonance system as claimed in claim 1, wherein the elastography apparatus comprises a holding apparatus on which the magnetic resonance-compatible motor is arranged during a magnetic resonance elastography examination.

6. The magnetic resonance system as claimed in claim 5, further comprising:
a patient support having a patient table that is movable within the patient receiving region,
wherein the holding apparatus is configured to be removably attached to the patient table.

7. The magnetic resonance system as claimed in claim 5, wherein:
the holding apparatus comprises a convex holding arc with two end regions and a central attachment region,
the two end regions are configured to be removably attached to a patient table, and
the central attachment region is configured to provide a location upon which the magnetic resonance-compatible motor is disposed.

8. The magnetic resonance system as claimed in claim 1, further comprising:
a positioning pad configured to position a patient,
wherein the magnetic resonance-compatible motor is arranged within the positioning pad.

9. The magnetic resonance system as claimed in claim 1, wherein the torque transmitter has a variable-length drive shaft.

10. The magnetic resonance system as claimed in claim 1, wherein the vibrator comprises a vibration element, and
wherein the drive torque generated by the magnetic resonance-compatible motor is transmitted to the vibration element.

11. The magnetic resonance system as claimed claim 1, wherein the vibrator comprises an eccentric element, and
wherein the drive torque generated by the magnetic resonance-compatible motor is transmitted to the eccentric element.

12. The magnetic resonance system as claimed in claim 1, wherein the elastography apparatus comprises a motor driver and a shield housing, and
wherein the motor driver is arranged in the shield housing.

13. The magnetic resonance system as claimed in claim 1, wherein the elastography apparatus comprises a controller configured to synchronize the elastography apparatus with a measurement sequence of the magnetic resonance elastography examination.

14. The magnetic resonance system as claimed in claim 13, wherein the elastography apparatus comprises a motor driver, and
wherein the elastography apparatus comprises an optical transmitter arranged between the motor driver and the controller.
